(19) United States
(12) Patent Application Publication
(10) Pub. No.: US 2005/0142204 A1
(43) Pub. Date: Jun. 30, 2005

(54) Triclosan dosage form

(76) Inventors: Antonie Philippus Lötter, Potchefstroom (ZA); Jan Lourens Du Preez, Potchefstroom (ZA); Lindi-May Collins, Potchefstroom (ZA)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/489,732

(22) PCT Filed: Sep. 18, 2002
(86) PCT No.: PCT/ZA02/00145

(30) Foreign Application Priority Data
Sep. 18, 2001 (ZA) 2001/7414

(51) Int. Cl. A61K 9/64; A61K 9/16; A61K 9/50

(52) U.S. Cl. 424/490; 424/456

(57) ABSTRACT

This invention relates to triclosan and more particularly to a dosage form of triclosan especially for use in the treatment, including prophylaxis, of malaria. This invention further relates to use of a triclosan emulsion or oil solution in the preparation of a composition for use in the treatment, including prophylaxis, of malaria. This invention also relates to a method of treating, including prophylaxis of malaria and the use of a triclosan emulsion or oil solution in such a method.

### FIELD OF THE INVENTION

This invention relates to triclosan and more particularly to a dosage form of triclosan especially for use in the treatment, including prophylaxis, of malaria. This invention further relates to use of triclosan in the preparation of a composition for use in the treatment, including prophylaxis, of malaria. This invention also relates to a method of treating, including prophylaxis, of malaria and the use of triclosan in such a method.

### BACKGROUND ART

Malaria remains a leading global health problem, despite considerable efforts to control the disease over several decades. Approximately 40% of the world's population live in malaria-endemic areas, with about 90% of cases and most deaths occurring in tropical Africa (Beeson et al., 2001:149). There are up to 500 million clinical cases and 2.7 million deaths, of which 1 million are child fatalities annually (WB, 2001). the majority of severe clinical disease is due to Plasmodium falciparum, the young children and pregnant women at highest risk (Beeson, et al., 2001:149).

Malaria also has a significant negative economic effect. Research shows that malaria-afflicted families are able to harvest only approximately 40% of their crops, compared with healthy families, suggesting a link between malaria and poverty. The direct and indirect costs of malaria in Africa alone are estimated to exceed US $2 billion per year, while it is believed that the disease could be controlled with a budget amounting to one-tenth of this amount. Malaria slows economic growth in African countries by an estimated 1.3% each year (MRC, 2001).

Malaria is caused by several species of the protozoan Plasmodium, of which P. vivas and P. falciparum are the most common. They all have complex life cycles involving both the Anopheles mosquito and the erythrocyte of the human host. In vivax, a persisting tissue phase continues to infect the blood at intervals for many years Thus, the ideal antimalarial should not only eradicate the microzoan from the blood, (i.e., to ‘suppress’ the clinical attack) but from the tissues as well, to effect a “radical cure”. The several antimalarials differ in their point of interruption of the cycle of the parasite and in the type of malaria affected (Harvey, 1975:1154).

Antimalarial treatment has advanced considerably over the last four centuries. Cinchona imported from Peru in 1643 allowed European countries and their colonies some means of suppressing the disease, and the introduction of quinine in the 19th century, followed by pamaquine in 1926 and quinacrine (alabrine) in 1930, improved treatment somewhat (Harvey, 1975:1154).

When supplies of quinine were cut off in World War II, the US Office of Scientific Research and Development co-ordinated a study of about 7000 new and an equal number of old, synthetic compounds. Not only were the older German compounds “rediscovered”, but also several new and superior agents (including amodiaquine, chloroquine, pentaquine, and primaquine) (Harvey, 1975:1154).

However, a major problem and disadvantage of the know drugs is the emergence and spread of antimalarial drug resistance. This makes the development of new drugs an important priority (Beeson et al., 2001:149). Resistance of the malaria parasite to chloroquine, one of the cheapest and previously most useful antimalarial agents, is now widespread. Similarly, resistance to the combination of sulphadoxine-pyrimethamine is extensive in Asia and growing in Africa. Resistance to quinene, the mainstay in treatment of severe disease, is becoming a major problem in certain parts of Asia. Relatively newer drugs, such as mefloquine, halofantrine, atovaquone-proguanil and artemether-lumefantrine still show efficacy but have limitations such as high cost. Novel uses for old drugs, such as chlorguanil-dapsone, and artemisinin combination therapy offer definite possibilities for the near future, but still have regulatory, policy and implementation hurdles to jump (Beeson et al., 2001:149).

Triclosan is a well known broad spectrum antibacterial agent active against many organisms. It has been in use as an antimicrobial agent in soaps, detergents, shampoos and various other household products for about 20 years. Extensive toxicity studies have been done, and it was proven to be safe topically as well as orally (Bhargava, H. & Leonard, P. A., Triclosan: Application and safety, American Journal of Infection Control, vol. 24, no. 3, June 1996).

Surolia (Surolia, N & Surolia A, Nature Medicine, vol. 7, no. 2 February 2001, p 167-173) showed that triclosan is active against malaria parasites. However, they state that it would be a long time before an oral dosage form is developed. It has been shown to be effective at a dose of 28-38 mg/kg. Beeson et al. (Beeson, J. G., Winstanely, P. A., McFadden, G. I. & Brown, G. V. New agents to combat malaria. Nature Medicine, vol. 7, no. 2 February 2001, p. 149-150) states that a lot of work is still to be done before the product will be of use. A major disadvantage of triclosan is its very low solubility in water which has a detrimental influence on its absorption and thus its bioavailability. Although extensive toxicity studies have been done, and triclosan has been proven safe for oral use, it has not yet been formulated for this route.

### OBJECTS OF THE INVENTION

It is therefore an object of the present invention to provide a dosage form of triclosan especially for the treatment, including prophylactic treatment, of malaria with which the aforesaid problems and disadvantages can be overcome or at least alleviated. Further objects of the invention are to provide use of triclosan in the preparation of a composition for use in the treatment, including prophylaxis, of malaria, a method of treating, including prophylaxis, of malaria and the use of triclosan in such a method, with which the aforesaid problems and disadvantages can be overcome or at least alleviated.

### SUMMARY OF THE INVENTION

According to the present invention there is provided use of a triclosan oil solution and/or triclosan emulsion in the preparation of a composition for use in the treatment, including prophylaxis, of malaria.

According to another aspect of the present invention there is provided a triclosan oil solution and/or triclosan emulsion for use in the treatment, including prophylaxis, of malaria.

According to yet another aspect of the present invention there is provided an anti-malaria dosage form comprising a triclosan oil solution and/or triclosan emulsion.

According to yet another aspect of the present invention there is provided the use of a triclosan oil solution and/or triclosan emulsion in the treatment, including prophylaxis, of malaria.

According to yet another aspect of the invention there is provided a method of manufacturing an anti-malaria dosage form including the steps of encapsulating triclosan in a form selected from the group consisting of an emulsion and an oil solution.

The triclosan may be dissolved or emulsified prior to encapsulation in a pharmacological acceptable oil selected from the group consisting of non-mineral oils, animal derived oils, plant derived oils, sunflower oil, olive oil, arachis oil, and sesame oil, and mixtures thereof.

The method may include the step of adding prior to encapsulation to the said triclosan form other formulation agents selected from the group consisting of anti-oxidants, BHA, surfactants, emulsifiers, preservatives, masking agents, sweeteners and combinations thereof.

According to yet another aspect of the present invention there is provided a method of treating a human or animal against malaria by administering a triclosan oil solution and/or emulsion to the human or animal. The treatment may also include prophylactic treatment.

In one embodiment of the invention the triclosan is provided in the form of a triclosan oil solution. The triclosan may be dissolved in any suitable pharmacological acceptable oil, preferably a non-mineral oil. The non-mineral oil may comprise an animal derived oil but preferably it comprises a plant, derived oil. The plant derived oil may comprise at least one oil of the group consisting of for example olive, arachis or sesame oil. Mixtures of the oils may also be used. In one embodiment of the invention sunflower oil may be used.

In another embodiment of the invention the triclosan may be provided in the form of a triclosan emulsion. Any suitable triclosan emulsion may be used. In one embodiment of the invention the emulsion comprises an oil-in-water emulsion. The oil may comprise any oil as defined above.

The triclosan oil solution and/or emulsion may be encapsulated, and this is especially the case where the triclosan is dissolved in a pharmacological acceptable oil. It appears that triclosan is very soluble in oils but has a bad taste at high concentration. The oily solution of triclosan may therefore not be acceptable to patients when administered as such and encapsulation should solve this problem. Preliminary studies have also shown that triclosan emulsions have a bad taste and that encapsulation of the emulsion may also be considered. The composition may be microencapsulated but preferably it is prepared as soft gelatin capsules.

The triclosan oil solution and/or emulsion may be taken orally and in such a case the dosage form preferably comprises an encapsulated triclosan oil solution and/or emulsion. It is believed that when administered orally especially as capsules, the triclosan oil solution and/or emulsion may be effectively absorbed via the lymph system.

The triclosan oil solution and/or suspension may also include other formulation agents. For example in the case where an oil is used as a solvent an anti-oxidant like BHA may be used to prevent oxidation of the oil. In the case of the triclosan suspension, surfactants, which serve as emulsifiers may be used. Preservatives and masking agents such as sweeteners may also be employed.

The invention will now be described further by means of the following non-limiting examples.

### EXAMPLE 1

### Triclosan Oil Solution for Encapsulation

Triclosan in the amount of 100 g was mixed with 200 g of sunflower oil with slight heating (up to 60° C.) until it dissolved. The solution was left to cool and de-aerate. Soft gelatin capsules of the triclosan oil solution were then prepared.

### EXAMPLE 2

### Triclosan Emulsion

The following compounds were used to prepare a high concentration triclosan emulsion:

| | |
|---|---|
| Triclosan | 16 g |
| Sunflower oil | 34 g |
| BHA | 0.01 g |
| Span 80 | 5 g |
| Tween 80 | 5 g |
| Methyl paraben | 0.1 g |
| Propyl paraben | 0.02 g |
| Na-saccharin | 0.1 g |
| Water qs to | 100 g |

BHA is an anti-oxidant and is added to prevent oxidation of the sunflower oil. Span 80 and Tween 80 are surfactants which serve as emulsifiers. Methyl paraben and propyl paraben are preservatives and Na-saccharin is s sweetener.

The triclosan was weighed and dissolved in the sunflower oil while stirring over low heat (up to 60° C.). When all the triclosan had dissolved, the BHA, Span 80, Tween 90 and preservatives were added. Na-saccharin was dissolved in a little warm water. If flavourants and colourants are used they may also be dissolved in the water. The water phase was then added to the oil phase with vigorous stirring (homogenizer) for emulsification. Water was added slowly up to volume.

### EXAMPLE 2

### The Assessment of the Bioavailability of Triclosan Dosage Forms

Method

Four volunteers were invited randomly to take part in the trial. The volunteers were in a rested and fasted state when the trial began. A single dose of 552 mg (two soft gelatine capsules) was administered and blood samples were drawn 15 times over 36 hours.

Quantitative analysis of the blood samples was conducted using an HPLC method developed and validated at the Research Institute for Industrial Pharmacy. Results of the analysis were evaluated statistically and values for AUC, Cₘₐₓ and tₘₐₓ were obtained.

HPLC Method for the Determination of Triclosan in Blood

A) Chromatographic Condition:
Analytical instrument: HP1050 series HPLC with a pump, autosampler, UV detector and Chemstation Rev. A 06.02 data acquisition and analysis software of equivalent.
Column: Luna C18-2 column, 150×4.6 mm, 5 μm
Mobile phase: Acentonitrile/water 70/30
Flow rate: 1.0 ml/min.
Injection volume: 100 μl.
Detection: UV at 210 nm.
Retention time: Approximately 5.1 and 6.3 minutes for triclosan and acenaphthene respectively.
Solvent: methanol.

B) Sample Preparation with enzymatic hydrolysis:
1. Pipet 1 ml of plasma into a 5 ml siliconised glass test tube.
2. Add 1 ml of 0.2 M sodium acetate-acetic acid buffer, pH 5.0.
3. Add 100 μl undiluted β-glucuronidase/arylsulfatase enzyme (Boehringer Mannheim 127 698).
4. Vortex mix for 10 seconds, seal and incubate at 40° C. for 12 hours.
5. Add 0.5 ml of the internal standard solution and centrifuge at 14000 rpm (4900 RCF) for 10 minutes before applying to the SPE columns.

C) Internal Standard Solution:
1. Weight approximately 5 mg of acenaphthene accurately and dissolve in 250 ml of solvent.
2. Add 0.5 ml of this solution to all standards and samples.

D) Standard Solution:
1. Weigh approximately 20 mg of triclosan accurately and dissolve in 250 ml of solvent.
2. Make further dilutions of 5 ml to 100 ml and 10 ml to 50 ml in water to obtain 80, 16- and 4.0 μg/ml stock solutions.

3. Use these solutions to spike blank plasma to obtain the following standards:

| Standard | Amount spiked | Concentration |
|---|---|---|
| solution (μg/ml) | (μl) | (ng/ml) |
| 4.0 | 10 | 40 |
| | 30 | 120 |
| 16 | 20 | 320 |
| | 40 | 640 |
| | 60 | 960 |
| | 80 | 1280 |
| 80 | 40 | 3200 |
| | 80 | 6400 |

Surolia and Surolia (2001:168) state that 3 μM (580 ng/ml) triclosan is sufficient for 50% inhibition of fatty acid synthesis in Plasmodium falciparum. One must assume that this includes triclosan in both the conjugated and unconjugated form.

E) Solid Phase Extraction:
Place the solid phase extraction (SPE) columns (Bond Elut C18, Varian, Harbor City, Calif.) onto a 16-position SPE vacuum manifold (Supelco, Bellefone, Pa.)
Prepare the SPE columns by passing 2 ml of methanol through them, followed by 1 ml of distilled water.
Apply the samples with the aid of an air displacement pipette.
Rinse the columns with 1 ml of distilled water.
Dry the SPE tubes by applying vacuum for about 2 minutes.
Elute the samples with 750 μl of methanol under very low vacuum (approximately 0.5 ml/minute flow rate) into 750 μl vials.
Inject into the chromatograph.

Results and Discussion

TABLE 1
Total triclosan in Plasma

| | subject no. | | | | |
|---|---|---|---|---|---|
| | 1 | 2 | 3 | 4 | Mean |
| Time (hours) | ng/ml | | | | |
| 0 | 0 | 0 | 0 | 0 | 0 |
| 0.333 | 248 | 1468 | 1622 | 1218 | 1139 |
| 0.667 | 547 | 9721 | 9366 | 2948 | 5646 |
| 1 | 765 | 12613 | 13980 | 11659 | 9754 |
| 1.333 | 869 | 12676 | 18210 | 14366 | 11530 |
| 1.667 | 1180 | 16978 | 15511 | 17817 | 12871 |
| 2 | 1422 | 16309 | 16833 | 15002 | 12391 |
| 2.5 | 3706 | 14937 | 22894 | 17633 | 14793 |
| 3 | 5144 | 22639 | 25263 | 17485 | 17633 |
| 4 | 4794 | 21800 | 19942 | 17107 | 15911 |
| 6 | 4267 | 17303 | 16072 | 11477 | 12280 |
| 8 | 3758 | 12911 | 15286 | 9083 | 10259 |
| 10 | 3376 | 10344 | 12462 | 8914 | 8774 |
| 12 | 2573 | 8932 | 8316 | 8115 | 6984 |
| 24 | 1565 | 6788 | 7663 | 5845 | 5465 |
| 36 | 567 | 3812 | 4086 | 4085 | 3137 |

TABLE 2
Statistical summary of results.

| Subject | AUC | Cₘₐₓ | Tₘₐₓ |
|---|---|---|---|
| 1 | 85654 | 5144 | 3.0 |
| 2 | 395120 | 22637 | 3.0 |
| 3 | 424825 | 25263 | 3.0 |
| 4 | 362079 | 17817 | 1.7 |
| Mean | 394008.0 | 21905.7 | 2.6 |
| SD | 25628.0 | 3083.5 | 0.6 |
| % RSD | 6.5 | 14.1 | 24.6 |

During the study, subjects felt comfortable at all times and never complained of experiencing any adverse effects (e.g. nausea, headaches or vomiting). No other side-effects were noted. The soft gelatine capsules may therefore be considered safe for oral administration.

As can be seen from tables 1 and 2 as well as FIG. 1, triclosan was released from the soft gelatine capsules and absorbed. The bioavailability was good in three of the four volunteers. The lower bioavailability in the fourth volunteer may be due to a number of circumstances, such as food intake, altered metabolic rate, and cannot be explained without further study.

[image]

### Conclusion

Triclosan concentrations as high as 22000 ng/ml (22 μg/ml) was found in plasma, which is about 30 times higher than the effective concentration mentioned by Surolia. The ingestion of the capsules did not result in any discomfort to the patients, and no adverse effects were reported. During a stability trial on the capsules, it was found to be stable, retaining 96% of its potency after 16 months.

The applicant has therefore found that triclosan in the form of any emulsion or an oil solution is an effective composition and dosage form for the treatment, including prophylaxis, of malaria. However, it will be appreciated that many variations in detail are possible with a dosage form of triclosan, the use of triclosan, a method of treating malaria according to the invention without departing from the scope of the appended claims. For example, the triclosan could be dissolved or emulsified in a pharmacological acceptable oil selected from the group consisting of non-mineral oils, animal derived oils, plant derived oils, sunflower oil, olive oil, arachis oil, and sesame oil, and mixtures thereof. Further for example, the triclosan form could include other formulation agents selected from the group consisting of anti-oxidants, BHA, surfactants, emulsifiers, preservatives, masking agents, sweeteners and combinations thereof. Even further for example, the triclosan form could be encapsulated or microencapsulated. Yet further for example, the triclosan form could be prepared in the form of soft gelatin capsules.

### References

1. BEESON, J. G., WINSTANLEY, P. A., MCFADDEN, G. I. & BROWN, G. V. 2001. New agents to combat malaria. Nature medicine (7(2):149-150, February.

2. HARVEY, S. C. 1975. Antimicrobial drugs.(In Hoover, J. E. et al., eds. Remington's pharmaceutical sciences. Easton, Pa. Mack Publishing Co. p.1123-1158.)

3. MRC (Medical Research Council, South Africa). 2001. General information on malaria. Available on Internet: http://www.malaria.org.za/Malaria_Risk/General_Information/general_inf ormation.htm

4. PORTER, C. J. H. 1999. Lipids, gastrointestinal uptake and drug absorption: in vivo and in vitro model selection. (In Barthelemy, P., ed. Recent advances in the formulation and development of poorly-soluble drugs. 33^{rd} Journeé Galéniques, 1999. Bulletin Technique Gattefossé no. 92. p. 21-28).

5. SCHULZE, J., MARQUARDT, F., LYMAN, F. & SPITZER, C. 1974. Determination of free and conjugated triclosan in blood by electron capture gas liquid chromatography. Journal of the American Oil Chemists' Society, 52:215-218.

6. SUROLIA, N. AND SUROLIA, A. 2001. Triclosan offers protection against blood stages of malaria by inhibiting enoyl-ACP reductase of Plasmodium falciparum. Nature medicine, 7(2):167-173, February.

7. WB (World Bank). 2001. Malaria at a glance. Available on Internet: http://www.rbm.who.int [Date of use: Aug. 24, 2001].

1-35. (canceled)

36. A method of manufacturing an oral anti-malaria dosage form including the steps of encapsulating triclosan in a form selected from the group consisting of an emulsion and an oil solution.

37. A method according to claim 36 wherein the triclosan is dissolved or emulsified prior to encapsulation in a pharmacological acceptable oil selected from the group consisting of non-mineral oils, animal derived oils, plant derived oils, sunflower oil, olive oil, arachis oil, and sesame oil, and mixtures thereof.

38. A method according to claim 36 or claim 37 including the step of adding prior to encapsulation to the said triclosan form other formulation agents selected from the group consisting of anti-oxidants, BHA, surfactants, emulsifiers, preservatives, masking agents, sweeteners and combinations thereof.

39. Triclosan in an encapsulated dosage form selected from the group consisting of an emulsion and an oil solution for use in the oral treatment, including prophylaxis, of malaria.

40. Triclosan according to claim 39 which is dissolved or emulsified in a pharmacological acceptable oil selected from the group consisting of non-mineral oils, animal derived oils, plant derived oils, sunflower oil, olive oil, arachis oil, and sesame oil, and mixtures thereof.

41. Triclosan according to claim 39 or claim 40 in combination with formulation agents selected from the group consisting of anti-oxidants, BHA, surfactants, emulsifiers, preservatives, masking agents, and sweeteners.

42. Triclosan according to claim 39 which is microencapsulated.

43. Triclosan according to claim 39 which is prepared as soft gelatin capsules.

44. An anti-malaria oral dosage form comprising triclosan in a form selected from the group consisting of an emulsion and an oil solution in a pharmaceutically acceptable encapsulation or micro-encapsulation.

45. An anti-malaria oral dosage form according to claim 44 wherein the triclosan is dissolved or emulsified in a pharmacological acceptable oil selected from the group consisting of non-mineral oils, animal derived oils, plant derived oils, sunflower oil, olive oil, arachis oil, and sesame oil, and mixtures thereof.

46. An anti-malaria dosage form according to claim 44 or claim 45 wherein the said triclosan form includes other formulation agents selected from the group consisting of anti-oxidants, BHA, surfactants, emulsifiers, preservatives, masking agents, sweeteners and combinations thereof.

47. A method of orally treating a human or animal against malaria by administering a pharmaceutically effective amount of triclosan in an oral dosage form selected from the group consisting of an emulsion and an oil solution to the human or animal.

48. A method according to claim 47 wherein the triclosan is dissolved or emulsified in a pharmacological acceptable oil selected from the group consisting of non-mineral oils, animal derived oils, plant derived oils, sunflower oil, olive oil, arachis oil, and sesame oil, and mixtures thereof.

49. A method according to claim 47 or claim 48 wherein the said triclosan form includes other formulation agents selected from the group consisting of anti-oxidants, BHA, surfactants, emulsifiers, preservatives, masking agents, sweeteners and combinations thereof.

50. A method according to claim 47 wherein the said triclosan form is microencapsulated.

51. A method according to claim 47 wherein the said triclosan form is prepared as soft gelatin capsules.

52. A method according to claim 47 which includes prophylactic treatment.
